(19) United States
(12) Patent Application Publication
(10) Pub. No.: US 2008/0085912 A1
(43) Pub. Date: Apr. 10, 2008

(54) Isoxazole derivatives and methods of treating diseases

(75) Inventors: Jiajiu Shaw, Ann Arbor, MI (US); An-Rong Lee, Taipei (TW); Wen-Hsin Huang, Taipei (TW)

Correspondence Address: BUTZEL LONG, 350 SOUTH MAIN STREET, SUITE 300, ANN ARBOR, MI, 48104, US

(73) Assignee: Unitech Pharmaceuticals, Inc.

(21) Appl. No.: 11/544,069

(22) Filed: Oct. 6, 2006

(51) Int. Cl. A61K 31/44; C07D 261/18

(52) U.S. Cl. 514/278; 548/248

(57) ABSTRACT

A series of isoxazole derivatives and methods of treating immune-mediated diseases by isoxazole derivatives are described.

### BACKGROUND

Leflunomide, N-(4-trifluoromethylphenyl)-4-carboxamidyl-5-methylisoxazole, has been shown to be effective in treating several immune-mediated diseases, including rheumatoid arthritis (RA), multiple sclerosis, and psoriasis. Leflunomide is an isoxazole derivative sold as Arava®.

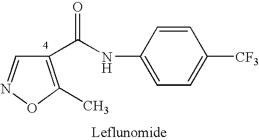

Valdecoxib, 4-(5-methyl-3-phenyl-4-isoxazolyl)benzenesulfonamide, has been used for the treatment of RA, osteoarthritis and dysmenorrhea pain. Valdecoxib is an isoxazole derivative sold as Bextra®.

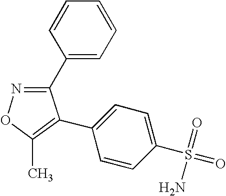

Some other isoxazole derivatives have also been shown to be effective as promising agents for treating RA and immune-mediated diseases. For example, our patented isoxazole derivatives (U.S. Pat. No. 6,727,272) have shown promising animal data in treating arthritis. Representative examples include N-(2-chlorophenyl)-3-carboxamidyl-5-methylisoxazole (or UTL-5b) and N-(4-chlorophenyl)-3-carboxamidyl-5-methylisoxazole (or UTL-5d).

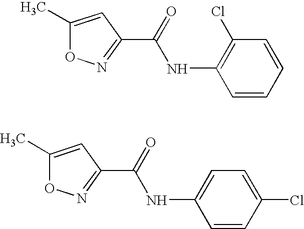

### SUMMARY

The scope of the present invention is defined solely by the appended claims, and is not affected to any degree by the statements within this summary.

By way of introduction, a compound embodying features of the present invention has a structure (I):

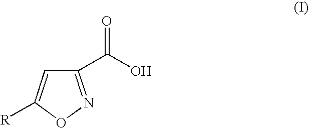

or a physiologically tolerable salt thereof; wherein —R is —H, -lower alkyl, —O-lower alkyl, -cyclyhexane, —CH₂-cyclohexane, —O-cyclohexane, —CH₂—O-cyclohexane, —C₆H₅, —CH₂—C₆H₅, —O—C₆H₅ or —CH₂—O—C₆H₅.

### BRIEF DESCRIPTION OF THE DRAWINGS

FIG. 1 shows a summary plot of Carrageenan-induced paw edema with pre-treatment of 5-metyhlisoxazole-3-carboxylic acid (UTR-1).

### DETAILED DESCRIPTION

Described hereinbelow is the surprising discovery of various isoxazole derivatives that can be used as agents for treating immune-mediated diseases, such as RA.

Because a number of isoxazole derivatives have shown to be effective in treating immune-mediated diseases, such as RA and multiple sclerosis, we theorized that there should be other isoxazole derivatives yet to be discovered as agents for treating immune-mediated diseases.

As part of our efforts in synthesizing and investigating isoxazole derivatives, we decided to test the effects of 5-methylisoxazole-3-carboxylic acid (UTR-1) in vivo and in vitro.

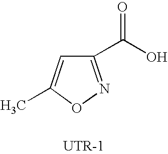

Results of a carrageenan-induced edema animal study indicate that UTR-1 is surpassingly effective in anti-inflammation (Example 1).

Results of an in vitro study show that UTR-1 is very effective in reducing TNF-α (tumor necrosis factor alpha) secreted from stimulated keratinocyte cells (Example 2). Results of the gene array analysis indicate that several immune related genes were significantly suppressed by UTR-1. The suppressed genes include Janus Kinase 3 (JAK3), mitogen activated protein kinase kinase kinase 2 (MAP3K2), etc. (Example 2).

JAK3 is a tyrosine kinase activated by interlukins IL-2, IL-4, IL-9, and IL-13. JAK3 serves in T cell activation and is associated with the hypersensitive response, severe combined immune deficiency (SCID), and likely atopic dermatitis. Protein serine/threonine kinase, MAP3K2, mediates T cell receptor activation of JNK signaling pathways; it activates NF-κB and may modulate immune and inflammatory responses.

In addition, a number of genes related to colon cancer were also significantly suppressed by UTR-1 (Example 2).

A first embodiment in accordance with the present invention is a compound having a general formula (I):

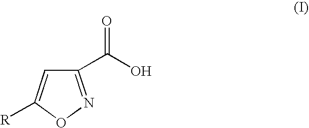

wherein —R is —H, -lower alkyl, —O-lower alkyl, -cyclyhexane, —CH₂-cyclohexane, —O-cyclohexane, —CH₂—O-cyclohexane, —C₆H₅, —CH₂—C₆H₅, —O—C₆H₅ or —CH₂—O—C₆H₅. As used herein, “lower alkyl” refers to a linear, branched or cyclic hydrocarbon containing from 2 to 5 carbons.

A second embodiment in accordance with the present invention is a pharmaceutical composition that comprises a compound of formula of (I) (or a physiologically tolerable salt thereof) and/or a compound of formula (II) (or a physiologically tolerable salt thereof):

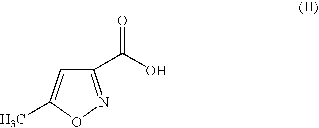

wherein —R is —H, -lower alkyl, —O-lower alkyl, -cyclyhexane, —CH₂-cyclohexane, —O-cyclohexane, —CH₂—O-cyclohexane, —C₆H₅, —CH₂—C₆H₅, —O—C₆H₅ or —CH₂—O—C₆H₅. “Lower alkyl” is as defined above. In some embodiments, the salt comprises sodium salt, ammonium salt or potassium salt.

Some representative examples of compounds embodying features of the present invention are shown below:

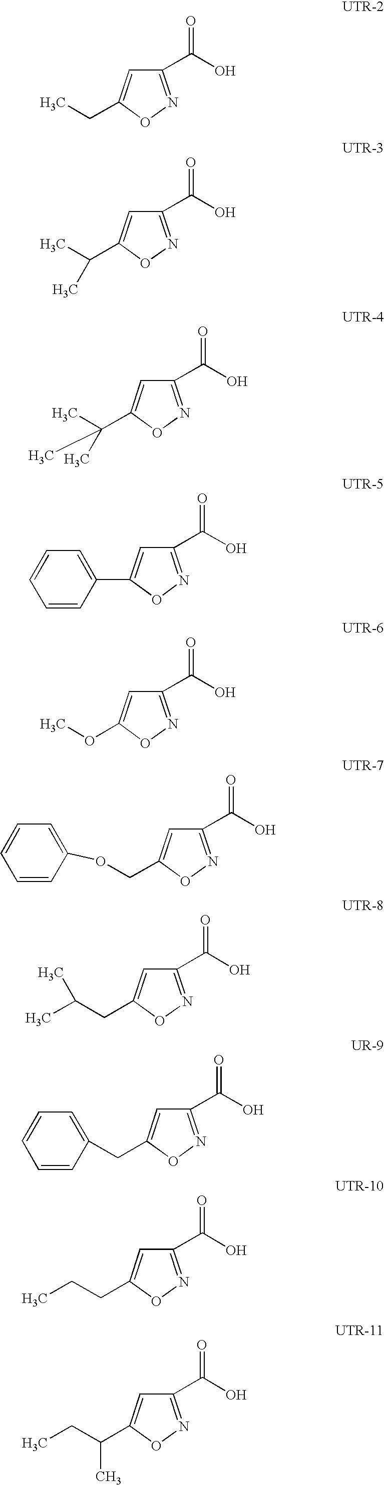

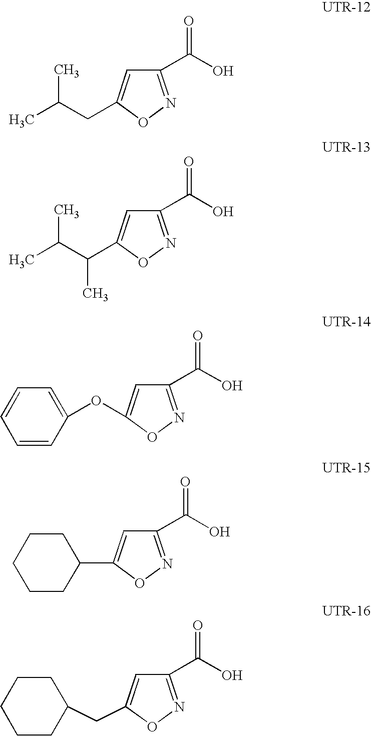

UTR-1 to UTR-6, UTR-8, and UTR-9 can be synthesized according to the following procedure:

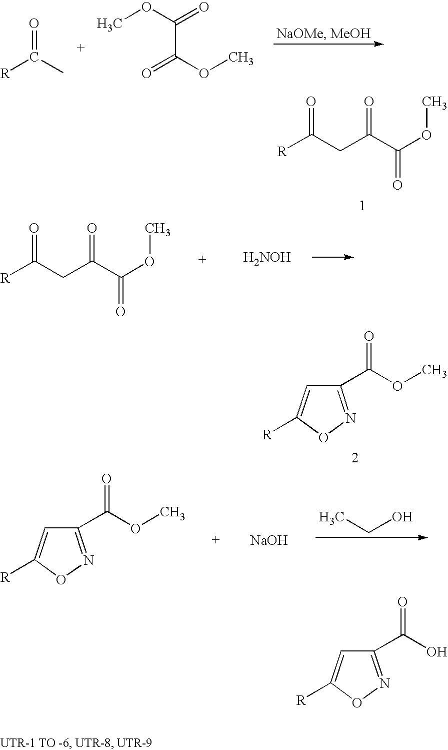

### UTR-7 can be synthesized according to the following scheme.

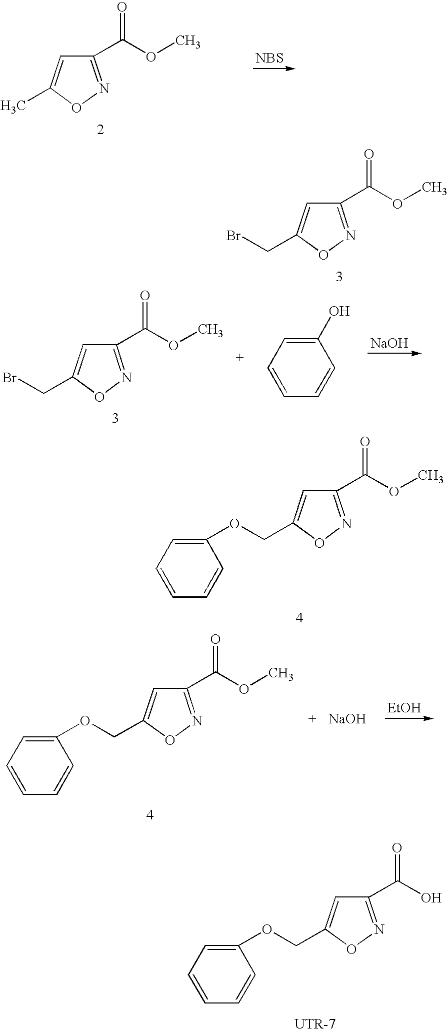

Examples of suitable forms of the pharmaceutical composition include but are not limited to tablets, coated tablets, solutions, suspensions, emulsions, powders, granules, (micro)capsules, suppositories, syrups, lotions, gels, creams, and the like, and combinations thereof.

A method of treating a patient having a TNF-α mediated disorder in accordance with the present invention comprises administering to a patient a therapeutically effective amount of the pharmaceutical composition. TNF-α mediated disorders include rheumatoid arthritis, psoriatic arthritis, Crohn's disease, psoriasis, lupus, atherosclerosis, scleroderma, multiple sclerosis, Alzheimer's disease, sepsis, type I diabetes, gingivitis, and cachexia.

A method of treating a patient having a JAK3 mediated disorder in accordance with the present invention comprises administering to a patient a therapeutically effective amount of the pharmaceutical composition. JAK3 mediated disorder include colon cancer, organ transplant rejection, psoriasis, and RA.

The following representative procedures and Examples are provided solely by way of illustration, and are not intended to limit the scope of the appended claims or their equivalents.

### EXAMPLE 1

### Determination of Anti-Inflammatory Activity of UTR-1 by the Carrageenan-Induced Hind Paw Edema Test on Rats

Male/female SD rats (180-215 g) were assigned to two groups: (1) control group and (2) test group, with 5 rats in each group. UTR-1 was first dissolved in DMSO (20 mg in 0.5 ml DMSO) and then diluted with 1% CMC (carboxymethylcellulose) to make 5 ml sample preparation.

One hour before the carrageenan challenge (pretreatment), 1 ml of the sample preparation was injected i.p. into a rat in a test group. This is equivalent to 20 mg/kg (1 ml×4 mg/ml×1000 g/200 g=20 mg/kg based on the average weight per rat of 200 g). The vehicle without drug was injected in the same way into animals in the control group.

In order to induce inflammation, 50 μl of a 1% carrageenan solution in normal saline was injected into the right hind paw subplantar tissue. The development of paw edema was measured by a plethysmometer (Basile 7140 plethysmometer, Ugo, Varese, Italy). After the carrageenan challenge, each paw volume (ml) was measured hourly.

The Edema (E %) is calculated as follows:

$$E \% = \frac{{V}_{1} - {V}_{0}}{{V}_{0}} \times 100 \%$$

V₀=Volume (mL) of the rear right footpad before the injection of Carrageenan

Vₜ=Volume (mL) of the rear right footpad at “t” time after the injection of Carrageenan

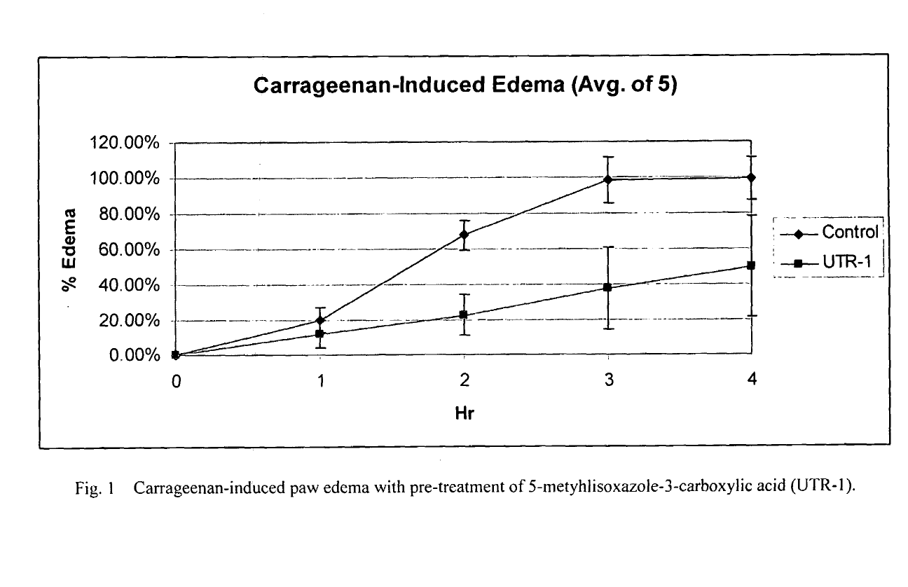

The results are shown in the following tables; a summary plot is shown in FIG. 1.

TABLE 1
Volume (ml) of the injected paw in Control Group

| | Time (hr) | | | | |
|---|---|---|---|---|---|
| | 0 | 1 | 2 | 3 | 4 |
| Rat 1 | 1.35 | 1.7 | 2.41 | 2.93 | 2.91 |
| Rat 2 | 1.25 | 1.53 | 2.19 | 2.45 | 2.56 |
| Rat 3 | 1.41 | 1.59 | 2.26 | 2.75 | 2.79 |
| Rat 4 | 1.24 | 1.57 | 2.04 | 2.49 | 2.42 |
| Rat 5 | 1.43 | 1.61 | 2.30 | 2.60 | 2.62 |

TABLE 2
Edema % of the injected paw in Control Group

| | Time (hr) | | | | |
|---|---|---|---|---|---|
| | 0 | 1 | 2 | 3 | 4 |
| Rat 1 | 0.00% | 25.93% | 78.52% | 117.04% | 115.56% |
| Rat 2 | 0.00% | 22.40% | 75.20% | 96.00% | 104.80% |
| Rat 3 | 0.00% | 12.77% | 60.28% | 95.04% | 97.87% |
| Rat 4 | 0.00% | 26.61% | 64.52% | 100.81% | 95.16% |
| Rat 5 | 0.00% | 12.59% | 60.84% | 81.82% | 83.22% |
| Avg E % | 0.00% | 20.06% | 67.87% | 98.14% | 99.32% |
| Stdev | 0.00% | 6.93% | 8.45% | 12.70% | 11.96% |

TABLE 3
Volume (ml) of the injected paw in UTR-1 treated group

| | Time (hr) | | | | |
|---|---|---|---|---|---|
| | 0 | 1 | 2 | 3 | 4 |
| Rat 1 | 1.48 | 1.47 | 1.53 | 1.55 | 1.62 |
| Rat 2 | 1.35 | 1.50 | 1.65 | 1.76 | 1.78 |
| Rat 3 | 1.33 | 1.53 | 1.66 | 2.10 | 2.35 |
| Rat 4 | 1.33 | 1.50 | 1.75 | 1.79 | 2.14 |
| Rat 5 | 1.46 | 1.77 | 1.92 | 2.36 | 2.49 |

TABLE 4
Edema % of the injected paw in UTR-1 treated group

| | Time (hr) | | | | |
|---|---|---|---|---|---|
| | 0 | 1 | 2 | 3 | 4 |
| Rat 1 | 0.00% | −0.68% | 3.38% | 4.73% | 9.46% |
| Rat 2 | 0.00% | 11.11% | 22.22% | 30.37% | 31.85% |
| Rat 3 | 0.00% | 15.04% | 24.81% | 57.89% | 76.69% |
| Rat 4 | 0.00% | 12.78% | 31.58% | 34.59% | 60.90% |
| Rat 5 | 0.00% | 21.23% | 31.51% | 61.64% | 70.55% |
| Avg E % | 0.00% | 11.90% | 22.70% | 37.85% | 49.89% |
| Stdev | 0.00% | 8.01% | 11.56% | 23.08% | 28.40% |

### EXAMPLE 2

### Modulation of TNF-α Released from Keratinocytes in vitro and Gene Array Analysis

### Test Materials

1. Vehicle stock: 50:50 v/v EtOH:PEG 600

2. UTR-1 stock: 3.5 mg/ml UTR-1 in a vehicle of 50:50 v/v EtOH:PEG 600

### Pretreatment

Human epidermal keratinocytes were seeded into 6-well plates and grown at 37±2° C. and 5±1% CO₂ using serum free Epilife media supplemented as recommended by the manufacturer. Upon reaching confluency, the media were removed and the cells were treated overnight with Epilife media containing 1% v/v each of the stock solutions above (Vehicle stock and UTR-1 stock). Final concentration was 35 μg/ml for UTR-1. Two wells in the 6-well plate were prepared for each treatment. After applying the test material the cells were incubated for 24 hours at 37±2° C. and 5±1% CO₂.

### Treatment and Microarray Analysis

At the end of the incubation period the culture media was removed via aspiration and replaced with phosphate buffered saline and the cells were then irradiated with ˜30 mJ/cm² of UVB radiation. After the irradiation, the phosphate buffered saline was replaced by fresh culture media and the cells were incubated overnight at 37±2° C. and 5±1% CO₂. After the incubation the cell culture media was collected to measure TNF-α release by a commercial ELISA Assay kit. The cells were washed once with PBS and then a trypsin/EDTA solution was added to release the cells, followed by the addition of trypsin neutralizing solution. The cells were collected and pooled into 15 ml centrifuge tubes based on their treatment and pelleted by centrifuging at 1000 RPM at 4±2° C. After removing the supernatant, the pelleted cells were lysed by adding 500 μl of guanidinium thiocyanate lysis solution to each tube and then repeatedly drawing and releasing the solution into the pipette until the cell pellet is dissolved. Total RNA was then isolated by an RNAqueous kit (Ambion). After purifying the RNA, mRNA is isolated and the converted into anti-sense RNA (aRNA). The aRNA is then labeled with a florescent probe. In this case, Cy3 (green florescent signal) is used to label the sRNA from untreated sample, while Cy5 (red fluorescent) is used to label the aRNA of treated sample. Once the aRNA is labeled and any unincorporated dye is removed from the sample, the labeled aRNA is mixed with a hybridized solution and applied to the microarray. The microarray is then hybridized overnight at 60-65° C. After hybridization, the microarray is washed to remove any unbound aRNA probe and then scanned with a microarray scanner. Criteria for evaluating changes in gene expression may vary. In general, (1) the fluorescence intensity of the gene marker should be greater than the background intensity, and (2) the ratio of Cy5/Cy3 (treated/untreated) fluorescence intensity needs to be greater than 1.3 or less than 0.66 to indicate a change of ±30% in gene expression.

### Results

The results of the TNF-α assay are presented in the following table. Values represent an n=2 (2 wells used for each treatment).

| | | | TNFα (pg/ml), | TNFα (pg/ml), | |
|---|---|---|---|---|---|
| Treatment | Abs 1 | Abs 2 | sample1 | Sample2 | Avg. |
| UVB + Vehicle | 0.304 | 0.172 | 257 | 147 | 202 |
| UVB + UTR-1 | 0.140 | 0.139 | 120 | 119 | 120 |

Results of the gene array analysis indicate that, among other genes, JAK3 and MAP3K2 are suppressed by UTR-1 as shown in the following table:

| | Ratio | |
|---|---|---|
| Gene | of treated/control | Suppression % |
| JAK3 (homo sapien) | 0.386 | 61% |
| JAK3 | 0.417 | 58% |
| MAP3K2 (homo sapien) | 0.592 | 40% |

The following genes related to colon cancer were also suppressed:

| Gene | Ratio of treated/control | Suppression % |
|---|---|---|
| PTGER1 | 0.286 | 71% |
| ITGB6 | 0.3 | 70% |
| APOBEC1 | 0.333 | 67% |
| TUCAN | 0.333 | 67% |
| EFA6R | 0.375 | 63% |
| DEFA6 | 0.38 | 62% |
| EPHB4 | 0.433 | 57% |

Description of each gene is shown in the table below:

| | |
|---|---|
| APOBEC1 | Apolipoprotein B mRNA editing enzyme catalytic polypeptide 1, catalyzes APOB mRNA |
| | editing by cytidine deaminase activity, resulting in the production of the truncated APOB |
| | isoform apoB-48; upregulated in some neurofibromatosis tumors and colon cancers |
| DEFA6 | Defensin alpha 6, an antimicrobial peptide that may be involved in host defense in the small |
| | bowel; elevated levels in the colon are associated with Crohns disease |
| EFA6R | ADP-ribosylation factor guanine nucleotide factor 6, a putative tumor antigen recognized by |
| | autoantibodies from hepatocellular carcinoma patients, expressed in colon cancer but not |
| | normal colon tissue |
| EPHB4 | Ephrin type B receptor 4, a member of the Eph-related subfamily of receptor tyrosine kinases, |
| | preferentially enhances megakaryocytic and erythrocytic differentiation of progenitor cells; |
| | expression is elevated in colon carcinoma |
| ITGB6 | Integrin beta 6, mediates epithelial cell-matrix interactions, development, wound repair, |
| | neoplasia, lung inflammatory response, squamous cell carcinoma invasion, proliferation of |
| | colon carcinoma cells, receptor for foot and mouth disease virus |
| PTGER1 | Prostaglandin E receptor subtype EP1, a G protein-coupled receptor that regulates intracellular |
| | calcium flux, progesterone biosynthesis; mouse Ptger1 may be involved in colon |
| | carcinogenesis |
| TUCAN | Tumor upregulated CARD-containing antagonist of caspase nine, member of the CARD |
| | family, inhibits the activation of several caspases as well as NF-kappaB, thus inhibiting |
| | apoptosis, overexpression in colon cancer correlates with shorter patient survival |

In conclusion, the present invention provides isoxazole derivatives for treating immune-mediated diseases. The results of our in vivo and in vitro studies indicate 5-methylisoxazole-3-carboxylic aicd is surprisingly and unexpectedly effective in anti-inflammation and in suppressing several immune related genes.

Although the description above contains many specificities, these should not be construed as limiting the scope of the invention but as merely providing the illustrations of some representative embodiments of this invention. Thus the scope of this invention should be determined by the appended claims and their legal equivalents, rather than by the description or examples given.

1. A compound having a structure (I): 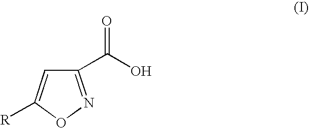
or a physiologically tolerable salt thereof,
wherein —R is —H, -lower alkyl, —O-lower alkyl, -cyclyhexane, —CH₂-cyclohexane, —O-cyclohexane, —CH₂—O-cyclohexane, —C₆H₅, —CH₂—C₆H₅, —O—C₆H₅ or —CH₂—O—C₆H₅.

2. A pharmaceutical composition comprising the compound of claim 1 or a compound having a structure (II): 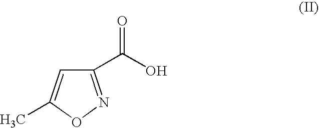
or a physiologically tolerable salt thereof.

3. The pharmaceutical composition of claim 2 wherein the salts comprise sodium salt, ammonium salt potassium salt or combinations thereof.

4. The pharmaceutical composition of claim 2 wherein the composition is formulated in a dosage form selected from the group consisting of tablets, coated tablets, injectable solutions, suspensions, emulsions, powders, granules, (micro)capsules, suppositories, syrups, lotions, gels, creams, and combinations thereof.

5. A method of treating a tumor necrosis factor alpha (TNF-α) mediated disorder comprising administering to a patient a therapeutically effective amount of the pharmaceutical composition of claim 2.

6. The method of claim 5 wherein the disorder is selected from the group consisting of rheumatoid arthritis, Crohn's disease, psoriasis, lupus, atherosclerosis, scleroderma, multiple sclerosis, Alzheimer's disease, sepsis, type I diabetes, gingivitis, cachexia, and combinations thereof.

7. A method of treating a Janus Kinase 3 (JAK3) mediated disorder comprising administering to a patient a therapeutically effective amount of the pharmaceutical composition of claim 2.

8. The method of claim 7 wherein the disorder is selected from the group consisting of colon cancer, psoriasis, organ transplant rejection, and combinations thereof.
